(19) United States
(12) Patent Application Publication
(10) Pub. No.: US 2008/0089848 A1
(43) Pub. Date: Apr. 17, 2008

(54) INTRATHECAL INJECTION OF MICROBUBBLES

(76) Inventor: Thomas DiMauro, Southboro, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/548,507

(22) Filed: Oct. 11, 2006

(51) Int. Cl. A61K 9/127

(52) U.S. Cl. 424/9.52; 424/450

(57) ABSTRACT

A method of delivering a therapeutic drug to a brain of a patient, involving intrathecally administering a microbubble composite containing a therapeutic agent, allowing the composite to rise into the cranium, and applying ultrasound to the cranium to explode the microbubbles.

### BACKGROUND OF THE INVENTION

According to Vink, Exp. Op. Invest. Drugs, October 2002, 11(1) 1375-86, and Vink, Exp. Op. Invest. Drugs, (2004) 13(10) 1263-74, “traumatic brain injury (TBI) is one of the leading causes of death and disability in the industrialized world and remains a major health problem with serious socioeconomic consequences. In industrialized countries, the mean incidence of traumatic brain injury (TBI) that results in a hospital presentation is 250 per 100,000. In Europe and North America alone, this translates to more than 2 million TBI presentations annually. Approximately 25% of these presentations are admitted for hospitalization. Those individuals who survive TBI are often left with permanent neurological deficits, which adversely affect the quality of life and as a result, the social and economic cost of TBI is substantial. Despite the significance of these figures, there is no single interventional pharmacotherapy that has shown efficacy in the treatment of clinical TBI.”

According to Vink, Exp. Op. Invest. Drugs, (2004) 13(10) 1263-74, “brain magnesium decline is a ubiquitous feature of TBI and is associated with the development of neurological deficits. Experimentally, parental administration of magnesium no more than 12 hours post-trauma restores brain magnesium homeostasis and profoundly improves both motor and cognitive outcome. While the mechanism of action is unclear, magnesium has been shown to attenuate a variety of secondary injury factors such as brain edema, cerebral vasopspasm, glutamate excitotoxicity, calcium-mediated events, lipid peroxidation, MPT and apoptosis.”

Despite the therapeutic properties of magnesium, the delivery of magnesium to the affected brain tissue remains an issue. For example, Brewer, Clin. Neuropharmacol., November-December, 2001, 24(6) 341-5 reported that systemic administration of magnesium sulfate failed to increase CSF ionized magnesium concentration in patients with intracranial hypertension despite increasing plasma magnesium levels by >50%. McKee, Crit. Care Med., March 2005 33(3) 661-6 investigated the brain bioavailability of peripherally administered magnesium sulfate, and reported that such hypermagnesia produced only marginal increases in total and ionized CSF [Mg]. McKee concluded that regulation of CSF [Mg] is largely maintained following acute brain injury and limits brain bioavailability of magnesium sulfate.

Buvanendran, Anesth. Analg., September 2002 95(3) 661-6 discloses administering intrathecal magnesium in order to prolong spinal opioid analgesia. However, large doses of intrathecal magnesium were not studied because of the limitations on cephalad spread when hyperbaric solutions are injected in the sitting position.

U.S. Pat. No. 6,123,956 (“Baker”) describes the intrathecal injection of therapeutic drugs as a way of bypassing the BBB. In general, Baker discloses encapsulating the drugs in microspheres, microcapsules, nanospheres and nanocapsules. Baker defines these carriers as having its encapsulated therapeutic agent material centrally located within a wall-forming polymeric material. Baker further teaches that polymeric microcapsules are preferably formed by dispersion of the therapeutic agent within liquified polymer, as described in U.S. Ser. No. 07/043,695, filed Apr. 29, 1987, U.S. Pat. No. 4,883,666 (“the Sabel patent”),

The Sabel patent teaches the encapsulation of therapeutic drugs within a polymeric device, wherein the outer wall of the polymeric device has a pinhole in order to produce linear release of the drug. The device of the Sabel patent generally requires the incorporation of an amount of therapeutic agent within an encapsulating polymer in an amount sufficient to produce an interconnected phase. This interconnected phase dissolves when contacted by water.

Freese, Exper. Neurology, 103, 234-8, 1989, which includes three co-inventors of the Sabel patent, is very similar to the Sabel patent in that it also discloses a device having a dopamine therapeutic agent encapsulated in an EVAc matrix. Freeze teaches that “although the polymer phase is impermeable to encapsulated molecules, release occurs as water enters the pore space, dissolving the solid particles. Molecules counterdiffuse out of the polymer through the pore network created by dissolution . . . Therefore, release of dopamine from the polymer must occur through a network of interconnected, aqueous pores. (See Freese at 236)”.

Therefore, the Sabel/Freeze technology as disclosed in the literature teaches an initially dense device that becomes porous upon contact with water and whose porosity fills with water in order to release the therapeutic agent.

The literature has also disclosed the intrathecal injection of liposomes containing local anaesthetics. For example, Umbrain, Acta Anaesthesiol. Scand., 1997, 41, 25-34 teaches the intrathecal injection of liposomes, and reports that the liposomes immediately diffused from the lumbar site of injection to the head. Umbrain reported that the 0.05 um smaller liposomes were rapidly absorbed into the blood via arachnoid granulations, while the larger 8 um liposomes could accumulate in the head with a slow elimination rate.

However, Umbrain's work was performed upon rat subjects, not humans. Whereas an intrathecally administered therapeutic need travel a few inches in a rat in order to reach the brain, an intrathecally administered therapeutic need travel a few feet in a human in order to reach the brain.

US Published Patent Application No. 2004/0105888 (“Pratt”) discloses buoyant polymer particles for delivery of therapeutic agents to the central nervous system, and compositions and methods for treating a subject who has suffered from a central nervous system disorder. More particularly, the invention provides sustained polymeric drug delivery systems for direct delivery of therapeutic agents into the central nervous system.

### SUMMARY OF THE INVENTION

The present invention relates to using microbubbles as carriers for intrathecally-injected therapeutic agents, such as magnesium sulfate. Because microbubbles have a porous core, they can be made to densities much lower than 1 g/cc. When such low densities are provided, these microbubbles become buoyant in water-based fluids, such as cerebrospinal fluid (CSF). The buoyancy of the microbubble allows the microbubble to rise through CSF. Therefore, a microbubble can be intrathecally injected into the CSF of a patient sitting in a prone position (such as through a lumbar puncture), and rise upward through the CSF, along the spinal canal and into the brain. Once in the brain, ultrasound may be applied to the microbubbles in order to burst the microbubble, thereby quickly releasing the therapeutic agent from within the microbubble.

Therefore, the present invention provides a directed flow of therapeutic agent to the brain while bypassing the blood brain barrier in a minimally invasive fashion.

Therefore, in accordance with the present invention, there is provided a method of delivering a therapeutic drug to a brain of a patient, comprising the step of:

a) intrathecally administering to the patient a microbubble composite comprising:
i) an outer wall section comprising a carrier and a therapeutic agent (preferably comprising magnesium), and
ii) a central substantially void section.

In preferred embodiments, the microbubbles have a small size. In this condition, they can pass through narrow regions of the spinal cord and brain without causing clogging. Therefore, in accordance with the present invention, there is provided a method of delivering a therapeutic drug to a brain of a patient, comprising the step of:
a) intrathecally administering a plurality of microbubbles comprising a therapeutic agent and a carrier to the patient,
b) allowing the composite to rise into the cranium, and
c) applying ultrasound to the cranium to explode the microbubbles.

### DETAILED DESCRIPTION OF THE INVENTION

In some embodiments, microbubbles are prepared by a double emulsion (W/O/W) solvent evaporation process. In general, a small amount of water is added to a larger amount of a non-polar liquid having the desired polymer dissolved therein. The water forms into spheres within the non-polar liquid phase. A much larger amount of water is then added to this mixture so that the non-polar liquid forms into bubble shapes. Next, the non-polar liquid is evaporated to harden the polymer. The capsules are then collected and the water in the internal portion of the capsule is evaporated to form the microbubble. The resulting product is a plurality of microbubbles having an external skin and an internal honeycomb structure.

In preferred embodiments thereof, the double emulsion is produced by following the teachings of El-Sherif, J. Biomed. Mat. Res., 66A:347-55, 2003. In particular, 0.5 g of PLGA is dissolved in 20 mL of methylene chloride. To generate the first W/O emulsion, 1.0 mL of deionized water is added to the polymer solution and probe sonicated at 110 W for 30 seconds. The W/O emulsion is then poured into a 5% PVA solution and homogenized for 5 minutes at 9500 rpm. The PVA acts as a surfactant and reduces the surface tension, whereas simultaneous homogenization breaks the W/O emulsion into a population of small beads. The double (W/O)/W emulsion is then poured into a 2% isopropanol solution and stirred at room temperature for 2 hours to evaporate off the methylene chloride and thus harden the capsules. The capsules are then collected by centrifugation, washed one time with deionized water, centrifuged at 15° C. for 5 minutes at 5000 g, and the supernatant is discarded. The capsules are then washed three times with hexane to further extract the methylene chloride from the polymer beads. The capsules are then frozen in a −85° C. freezer and lyophilized using a freeze dryer to fully dry the capsules and sublime the encapsulated water. This method produces microbubbles having an average diameter of about 1.1 μm.

Assuming the PLGA in the El-Sherif method has a density of about 1.4 g/cc, the 0.5 g PLGA phase will have a volume of about 0.33 cc. Because water has a density of 1.0 g/cc, the aqueous phase will have a volume of 1 cc. Thus, the total volume of the microbubbles provided by this method should be about 1.33 cc. The weight of the microbubbles should be about 0.5 g PLGA. Therefore, the density of the microbubbles should be 0.5 g/1.33 cc, or about 0.4 g/cc. Thus, the PLGA microbubbles should be highly buoyant.

In some embodiments, the therapeutic agent is added to the PLGA phase prior to formation of the W/O emulsion (or after the PLGA is dissolved in the methylene chloride and before addition of the PVA to make the double emulsion). This results in the therapeutic agent being present within the polymer phase. The therapeutic agent is typically released from this phase in two phases. The first phase, the burst phase, typically releases 10-30% of the therapeutic agent. The second phase, the slow release phase, releases the polymer degrades (or, if present in such large quantities as to form a continuous interdigitated phase within the polymer, by its dissolution in the CSF).

In some embodiments, the therapeutic agent is added to the deionized water that is used to generate the first W/O emulsion. This results in the therapeutic agent being present within the porosity of the microbubble (i.e., being encapsulated in the carrier (e.g., PLGA) polymer. The therapeutic agent is release from this phase by ultrasound-mediated destruction of the microbubble.

The density of the microbubble is essentially determined by the water: polymer ratio. In general, the lower the density of the composite, the more buoyant the composite, and the more quickly the composite will rise through the spinal canal to the patient's head. In some embodiments, the composite has a density of less than 0.8 g/cc, preferably less than 0.6 g/cc, more preferably less than 0.4 g/cc.

Preferably, the composite of the present invention has a diameter of not more than 100 um. When the composite has a diameter of not more than 100 um, there is a reduced chance that the composite may get stuck. More preferably, the composite of the present invention has a diameter of not more than 50 um, still more preferably not more than 20 um.

Also preferably, the composite of the present invention has a diameter of at least 0.05 um. When the composite of the present invention has a diameter of at least 0.05 um, it will clear less rapidly from the head through the arachnoid villae, thereby allowing for sustained release of the therapeutic agent. More preferably, the composite of the present invention has a diameter of at least 0.5 um, more preferably at least 8 um.

In some embodiments, the composite of the present invention has a diameter of between 8 um and 20 um. In this range, the composite is large enough to avoid rapid clearance via arachnoid villae, and yet small enough to avoid getting stuck.

In some embodiments, the microbubbles are nitrogen-filled. This requires that the sublimation step be carried out in an atmosphere consisting essentially of nitrogen. In some embodiments, the microbubbles are perfluorocarbon-filled. This requires that the sublimation step be carried out in an atmosphere consisting essentially of perfluorocarbon.

The therapeutic agents that may be beneficially delivered to the brain include but are not limited to magnesium compounds, EPO, anti-excitotoxic compounds (such as lubeluzole), neurotrophins, growth factors, agents that bind to beta amyloid protein with high affinity, and anti-inflammatory compounds.

The magnesium compound can be selected from the group consisting of magnesium metal, magnesium oxide, magnesium stearate, magnesium citrate, magnesium chloride, magnesium sulfate, magnesium carbonate, magnesium hydroxide, magnesium gluconate, magnesium phosphate and magnesium aspartate.

When MgO is selected as the magnesium compound, it is preferably provided in the form of a powder, as MgO is more readily hydrolysable when in a powder form. When MgO is hydrolyzed, it forms Mg(OH)₂, which solubilizes to Mg⁻² cations and OH⁻ anions. Since it is known that, during brain injury, the pH of brain tissue decreases (Gupta, J. Neurotrauma, June 2004, 21(6) 678-84), the release of hydroxide ions from magnesium hydroxide may beneficially restore the pH of brain tissue.

In some embodiments wherein Mg(OH)₂ is selected as the magnesium compound, it is provided in a PLGA carrier in accordance with the techniques described in Aubert-Pouessel, Brain Res. July 2002, 19,7,1046-51

In some embodiments, MgCl₂ is selected as the magnesium compound. In some preferred embodiments thereof, MgCl₂ is loaded into a polyethylene vinyl acetate (PEVAc) carrier in accordance with the techniques disclosed in Vasudev, Drug Delivery, 6, 117-126 (1999). The cerebral use of ethylene vinyl acetate compounds as drug carriers has been investigated by Wiranowska, J. Interferon Cytokine Res., June 1998, 18(6) 377-85; Saltzman, Pharm. Res., February 1999, 16(2) 232-40; Tomqvist, Exp. Neurology, 164, 130-138(2000); Pradilla, J. Neurosurgery, July 2004, 101(1) 99-103.

In some preferred embodiments thereof, MgCl₂ is loaded into a PLGA carrier in accordance with the techniques disclosed in Shenderova, Pharm. Res., Feb. 16(2) 241-8.

In some preferred embodiments thereof, MgCl₂ is loaded into a dextran-PEG 400 carrier in accordance with the techniques disclosed in Bronsted, J. Controll. Rel., 53 (1998) 7-13.

In some embodiments, MgCO₃ is selected as the magnesium compound. In preferred embodiments thereof, MgCO₃ is loaded into a PLGA carrier in accordance with the techniques disclosed in Sandor, Biochim. Biophys. Acta., February 2002, 15, 1570(1) 63-74.

In addition to TBI, it is believed that the methods and devices of the present invention could be useful in increasing the magnesium level in the brain of a stroke patient, or in the brain of an epileptic patient, or in the brain of a patient having Parkinson's Disease (PD), or in the brain of a patient having a migraine headache. Muir, Postgrad Med. J. 202, 78, 641-645 reports that systemically administered magnesium is useful in treating stroke.

In some embodiments, magnesium sulfate (MgSO₄) is used as the magnesium compound, and is provided in the first aqueous phase of the W/O/W emulsion. In this embodiment, the MgSO₄ dissolves in the aqueous phase, and is re-precipitated upon evaporation of the water during the lyophilization step, so that it is essentially encapsulated as a solid phase within the porosity of the PLGA microbubble. Upon lumbar injection and transport up to the cranium, these microbubbles will be exploded by application of transcranial ultrasound and the MgSO₄ will be released from the microbubbles, exposed to the aqueous-based CSF, and dissolved therein.

The following will demonstrate that addition of a therapeutic amount of MgSO₄ to an appropriate amount of microbubbles will result in a porous, buoyant therapeutic product that can be provided to the patient on a daily basis:

According to Muir, Postgrad Med. J. 202, 78, 641-645, the average magnesium concentration in human cerebrospinal fluid is about 1.1 mM. If a TBI patient has a reduced Mg level in the CSF of about 0.6 mM, then it would be desirable to add an increment of about 0.5 mM to the magnesium concentration in the CSF. Since MgSO₄ has a molecular weight of about 120, it would be desirable to add a concentration increment of about 60 mg/L to the CSF. Since CSF turns over at a rate of about 500 mL per day, the physician would want to add about 30 mg MgSO₄ per day to the CSF to this patient in order to attain the 1.1 nM magnesium goal.

Assuming that the patient is provided with one injection of MgSO₄-laden microbubbles per day of microbubbles manufactured via the El-Sherif W/O/W method described above, the El-Sherif method would be followed, except that 30 mg of MgSO₄ would be added to the 1 ml of deionized water to produce the desired daily formulation.

Assuming the PLGA has a density of about 1.4 g/cc, the 0.5 g PLGA phase will have a volume of about 0.33 cc. Because water has a density of 1.0 g/cc, the aqueous phase will have a volume of 1 cc. Thus, assuming negligible MgSO₄ volume, the total volume of the microbubbles provided to the patient on a daily basis should be about 1.33 cc. The weight of the microbubbles should be about 0.5 g PLGA+0.030 g MgSO₄/cc. Therefore, the density of the microbubbles should be 0.53 g/1.33 cc, or about 0.4 g/cc. Thus, the MgSO₄-laden microbubbles should be highly buoyant.

In some embodiments, the therapeutic agent contained in the microbubble is erythropoietin (EPO).

In some embodiments, EPO is provided in the polymer phase of the W/O/W emulsion. Upon lumbar injection and transport up to the cranium, the EPO in these microbubbles will be released in an initial burst phase and then in a slow release phase.

It is further believed that the small amounts of EPO provided in the microbubbles will have a negligible effect upon the buoyancy of the microbubbles.

Natural or native Erythropoietin is a 30-kDa glycoprotein that controls erythropoiesis by regulating the differentiation, proliferation and survival of erythroid precursor cells (3). As used herein and as defined within the claims, the term “EPO” shall include those polypeptides and proteins that have the capacity to stimulate erythropoiesis as mediated through the native Erythropoietin receptor. The term “EPO” includes natural or native erythropoietin as well as recombinant human erythropoietin (r-HuEPO). Also included within the scope of the term EPO are erythropoietin analogs, erythropoietin isoforms, erythropoietin mimetics, erythropoietin fragments, hybrid erythropoietin proteins, fusion protein oligomers and multimers of the above, homologues of the above, glycosylation pattern variants of the above, peptide mimetics and muteins of the above, and further regardless of the method of synthesis or manufacture thereof including, but not limited to, recombinant (whether produced from cDNA or genomic DNA), synthetic, transgenic, and gene activated methods, and further those Erythropoietin molecules containing the minor modifications enumerated above. Methods of designing and synthesizing, e.g., peptide mimetics are well known to those of ordinary skill in the art and are described, e.g., in U.S. Pat. Nos. 4,833,092, 4,859,765; 4,853,871 and 4,863,857 the disclosures of each of which are hereby incorporated by reference herein in their entirety and for all purposes. In addition to polypeptides and proteins having erythropoietic activity, small molecules capable of promoting erythropoiesis are also within the scope of the term EPO and include, for example, compounds with erythropoietin activity, such as molecules that stimulate erythropoietin production through upstream activation events.

Particularly preferred EPO molecules are those that are capable of stimulating erythropoiesis in a mammal. Specific examples of erythropoietin include, Epoetin alfa (EPREX.RTM., ERYPO.RTM., PROCRIT.RTM.), novel erythropoiesis stimulating protein (NESP.™., ARANESP.™. and darbepoetin alfa) such as the hyperglycosylated analog of recombinant human erythropoietin (Epoetin) described in European patent application EP640619. Other EPO molecules contemplated within the scope of the invention include human erythropoietin analogs (such as the human serum albumin fusion proteins described in the international patent application WO 99/66054), erythropoietin mutants described in the international patent application WO 99/38890, erythropoietin omega, which may be produced from an Apa I restriction fragment of the human erythropoietin gene described in U.S. Pat. No. 5,688,679, altered glycosylated human erythropoietin described in the international patent application WO 99/11781 and EP1064951, PEG conjugated erythropoietin analogs described in WO 98/05363, WO 01/76640, or U.S. Pat. No. 5,643,575. Specific examples of cell lines modified for expression of endogenous human erythropoietin are described in international patent applications WO 99/05268 and WO 94/12650. The generally preferred form of EPO is purified recombinant human EPO (r-HuEPO), currently formulated and distributed under the trademarks of EPREX.RTM., ERYPO.RTM., PROCRIT.RTM. or ARANESP.™. The disclosures of each of the patents and published patent applications mentioned in this paragraph are hereby incorporated by reference herein for any and all purposes.

Long-acting forms of EPO are also contemplated and may be preferred in some embodiments of the present invention for administration as the second or third exposure in a dosing segment. As used herein, a “long-acting EPO” includes sustained-release compositions and formulations of EPO with increased circulating half-life, typically achieved through modification such as reducing immunogenicity and clearance rate, and EPO encapsulated in polymer microspheres. Examples of “long-acting EPO” include, but are not limited to, conjugates of erythropoietin with polyethylene glycol (PEG) disclosed in PCT publication WO 2002049673 (Burg et al.), PEG-modified EPO disclosed in PCT publication WO 02/32957 (Nakamura et al.), conjugates of glycoproteins having erythropoietic activity and having at least one oxidized carbohydrate moiety covalently linked to a non-antigenic polymer disclosed in PCT publication WO 94/28024 (Chyi et al.), and other PEG-EPO prepared using SCM-PEG, SPA-PEG AND SBA-PEG. The disclosures of each of these published patent applications are hereby incorporated by reference herein in their entirety and for all purposes.

In some embodiments, the microbubble contains both EPO and a TNF-α antagonist.

Without wishing to be tied to a theory, it is believed that the survival-promoting effects of EPO are carried out through activation of phosphatidylinositol 3′-kinase (PI 3-kinase), and that TNF-α at low concentrations inhibit an essential component of the EPO survival response, namely activation of phosphatidylinositol 3′-kinase (PI 3-kinase). Therefore, it would be useful to combine the survival-promoting effects of EPO with a TNF-α antagonist. In some embodiments wherein a stroke has significantly compromised the blood brain barrier, EPO is administered intravenously and a plurality of microbubbles containing a TNF-α antagonist is administered intrathecally.

Concentrations of TNF-α as low as 10 pg/ml markedly reduce the capacity of IGF-I to promote survival of primary murine cerebellar granule neurons. Venters, PNAS USA, 96,9879-84, August 1999.

According to Marranness, J. Pharmacol. Exp. Therapeutics, 295(2) 2000, 531-545, lubeluzole is the (+)-S enantiomer of a benzothiazole derivative that has a neuroprotective action in animal models of focal and global ischemia, in which it reduces sensormotor deficits and the infarct volume. Lubeluzole inhibits glutamate-induced nitric oxide related neurotoxicity and blocks neurotoxicity induced by nitric oxide donors. Because of these qualities, lubeluzole has been proposed as a therapeutic in early stage ischemic stroke. However, maintenance of adequate CNS levels of lubeluzole has been found to be problematic.

The present inventors have developed inventions for treating brain injury and stroke with a composite comprising a plurality of microbubbles containing a thiazole-containing compound.

In some embodiments, the composite in administered intrathecally (such as through a lumbar puncture) and then buoyantly lifted upward through the spinal CSF and into the cranium to the site of the brain injury. Once sited at the location of the injury, the thiazole-containing compound is released from the composite and ameliorates the stroke.

Preferably, the thiazole-containing compound is a benzothiazole, more preferably lubeluzole.

In some embodiments, lubeluzole is delivered to the brain via in microbubbles comprising PEG. PEG appears to be the primary ingredient of the oral drops of Example 12 of U.S. Pat. No. 5,434,168 (“the lubeluzole patent”), the specification of which is incorporated by reference in its entirety.

Briefly, 50 g of lubeluzole is dissolved in 0.5 liters of 2-hydroxypropanoic acid and 1.5 liters of the polyethylene glycol (PEG) at about 60° C.-80° C. After cooling to about 30° C.-40° C., there are added about 35 liters of polyethylene glycol and the mixture is stirred well. Polyethylene glycol is then added to a volume of 50 liters providing a solution comprising 1 mg/ml of lubeluzole. The resulting solution is added to a non-polar liquid.

In some embodiments, lubeluzole is delivered to the brain via a cellulose carrier. Cellulose is frequently cited as a carrier for benzothiazole derivatives, and is listed as an ingredient in the tablet example 15 of the lubeluzole patent.

The present inventors have further developed inventions for treating neurodegenerative disease with a composite comprising a microbubble and a neurotrophin.

In preferred embodiments, the composite in administered intrathecally (such as through a lumbar puncture) and then buoyantly lifted through the spinal CSF and into the cranium to the site of the brain injury. Once sited at the location of the injury, the neurotrophin is released from the composite and ameliorates the disease.

In some embodiments, the neurotrophin is NGF. When NGF is selected as the neurotrophin, it may be combined with a PLGA carrier. Techniques for providing NGF in a PLGA carrier are disclosed in Camarata, Neurosurg., March 1992, 30, 3,313-319, and in Hadlock, J. Reconstr. Microsurg., April 2003, 19(3), 179-84, 185-6.

In some embodiments, the neurotrophin is BDNF. When BDNF is selected as the neurotrophin, it may be combined with a PLGA carrier. Techniques for providing BDNF in a PLGA carrier are disclosed in Mittal, Neuroreport., December 1996, 20, 5, 18,2577-82.

In some embodiments, the neurotrophin is CTNF. When CTNF is selected as the neurotrophin, it may be combined with a PLGA carrier. Techniques for providing CTNF in a PLGA carrier are disclosed in Maysinger, Exp. Neurol., April 1996, 138(2) 177-188.

In some embodiments, the neurotrophin is GDNF. When GDNF is selected as the neurotrophin, it may be combined with a PLGA carrier. Techniques for providing GDNF in a PLGA carrier are disclosed in Aubert-Pouessel, J. Controll. Release, March 2004, 24, 95,3,463-475.

The present inventors have further developed inventions for treating neurodegenerative disease with a composite comprising a microbubble containing a growth factor.

In preferred embodiments, the composite in administered intrathecally (such as through a lumbar puncture) and then buoyantly lifted through the spinal CSF and into the cranium to the site of the brain injury. Once sited at the location of the injury, the growth factor is released from the composite and ameliorates the disease.

In some embodiments, the growth factor is IGF-I. When IGF-I is selected as the growth factor, it may be combined with a PLGA carrier. Techniques for providing IGF-I in a PLGA carrier are disclosed in Carrascosa, Biomaterials, February 2004, 25, 4, 707-714.

In some embodiments, the growth factor is VEGF. The literature reports that VEGF can be neuroprotective in both Alzheimer's Disease (AD) and Parkinson's Disease (PD). Yang, J. Neurochem., April 2005, 93(1) 118-127 reports that VEGF provides neuroprotection in AD by binding to beta amyloid protein. Yasuhara, Brain Research, March 2005, 15, 1038, 1, 1-10 reports that low dose VEGF is neuroprotective towards dopaminergic neurons in PD.

When VEGF is selected as the growth factor, it may be combined with a PLGA carrier. Techniques for providing VEGF in a PLGA carrier are disclosed in Faranesh, Magn. Reson. Med., June 2004, 51,6,1265-1271.

In some embodiments, the growth factor is a BMP. BMPs disclosed in U.S. Pat. No. 6,936,582, the specification of which is incorporated by reference in its entirety, are contemplated for use in the present invention.

The OP/BMP morphogens of the present invention are naturally occurring proteins, or functional variants of naturally occurring proteins, in the osteogenic protein/bone morphogenetic protein (OP/BMP) family within the TGF-β superfamily of proteins. That is, these proteins form a distinct subgroup, referred to herein as the “OP/BMP morphogens,” within the loose evolutionary grouping of sequence-related proteins known as the TGF-β superfamily. Members of this protein family comprise secreted polypeptides that share common structural features, and that are similarly processed from a pro-protein to yield a carboxy-terminal mature protein. Within the mature protein, all members share a conserved pattern of six or seven cysteine residues defining a 97-106 amino acid domain, and the active form of these proteins is either a disulfide-bonded homodimer of a single family member, or a heterodimer of two different members. See, e.g., Massague, Annu. Rev. Cell Biol. 6:597 (1990); Sampath et al., J. Biol. Chem. 265:13198 (1990). For example, in its mature, native form, natural-sourced human OP-1 is a glycosylated dimer typically having an apparent molecular weight of about 30-36 kDa as determined by SDS-PAGE. When reduced, the 30 kDa protein gives rise to two glycosylated peptide subunits having apparent molecular weights of about 16 kDa and 18 kDa. The unglycosylated protein has an apparent molecular weight of about 27 kDa. When reduced, the 27 kDa protein gives rise to two unglycosylated polypeptide chains, having molecular weights of about 14 kDa to 16 kDa.

Typically, the naturally occurring OP/BMP proteins are translated as a precursor, having an N-terminal signal peptide sequence, a “pro” domain, and a “mature” protein domain. The signal peptide is typically less than 30 residues, and is cleaved rapidly upon translation at a cleavage site that can be predicted using the method of Von Heijne, Nucleic Acids Research 14:4683-4691 (1986). The “pro” domain is variable both in sequence and in length, ranging from approximately 200 to over 400 residues. The pro domain is cleaved to yield the “mature” C-terminal domain of approximately 115-180 residues, which includes the conserved six- or seven-cysteine C-terminal domain of 97-106 residues. As used herein, the “pro form” of an OP/BMP family member includes a protein comprising a folded pair of polypeptides, each comprising a pro domain in either covalent or noncovalent association with the mature domains of the OP/BNP polypeptide. Typically, the pro form of the protein is more soluble than the mature form under physiological conditions. The pro form appears to be the primary form secreted from cultured mammalian cells. The “mature form” of the protein includes a mature C-terminal domain which is not associated, either covalently or noncovalently, with the pro domain. Any preparation of OP-I is considered to contain mature form when the amount of pro domain in the preparation is no more than 5% of the amount of “mature” C-terminal domain.

OP/BMP family members useful herein include any of the known naturally-occurring native proteins including allelic, phylogenetic counterpart and other variants thereof, whether naturally-sourced or biosynthetically produced (e.g., including “muteins” or “mutant proteins”), as well as new, active members of the OP/BMP family of proteins.

Particularly useful sequences include those comprising the C-terminal seven cysteine domains of mammalian, preferably human, human OP-1, OP-2, OP-3, BMP2, BMP3, BMP4, BMP5, BMP6, BMP8 and BMP9. Other proteins useful in the practice of the invention include active forms of GDF-5, GDF-6, GDF-7, DPP, Vg1, Vgr-1, 60A, GDF-1, GDF-3, GDF-5, GDF-6, GDF-7, BMP10, BMP11, BMP13, BMP15, UNIVIN, NODAL, SCREW, ADMP or NURAL and amino acid sequence variants thereof. In one currently preferred embodiment, the OP/BMP morphogens of the invention are selected from any one of: OP-1, OP-2, OP-3, BMP2, BMP3, BMP4, BMP5, BMP6, and BMP9.

Publications disclosing these sequences, as well as their chemical and physical properties, include: OP-1 and OP-2: U.S. Pat. No. 5,011,691, U.S. Pat. No. 5,266,683, and Ozkaynak el al., EMBO J. 9:2085-2093 (1990); OP-3: WO94/10203; BMP2, BMP3, and BMP4: U.S. Pat. No. 5,013,649, WO91/18098, WO88/00205, and Wozney et al., Science 242:1528-1534 (1988); BMP5 and BMP6: WO90/11366 and Celeste et al., Proc. Natl. Acad. Sci. (USA) 87:9843-9847 (1991); Vgr-1: Lyons et at., Proc. Natl. Acad. Sci. (USA) 86:4554-4558 (1989); DPP: Padgett et al., Nature 325:81-84 (1987); Vgl: Weeks, Cell 51:861-867 (1987); BMP9: WO95/33830; BMP10: WO94/26893; BMP-11: WO94/26892; BMP12: WO95/16035; BMP-13 WO95/16035, GDF-1: WO92/00382 and Lee et al., Proc. Natl. Acad. Sci (USA) 88:4250-4254 (1991); GDF-8: WO94/21681; GDF-9: WO94/15966; GDF-10: WO95/10539; GDF-11: WO96/01845; BMP-15: WO96/36710; MP121: WO96/01316; GDF-5 (CDMP-1, MP52): WO94/15949, WO96/14335, WO93/16099 and Storm el al., Nature 368:639-643 (1994); GDF-6 (CDMP-2, BMP13): WO95/01801, WO96/14335 and WO95/10635; GDF-7 (CDMP-3, BMP12): WO95/10802 and WO95/10635; BMP-3b: Takao et al., Biochem. Biophys. Res. Comm. 219:656-662 (1996); GDF-3: WO94/15965; 60A: Basler et al., Cell 73:687-702 (1993) and GenBank Accession No. L12032. In another embodiment, useful proteins include biologically active biosynthetic constructs, including novel biosynthetic proteins and chimeric proteins designed using sequences from two or more known OP/BNT family proteins. See also the biosynthetic constructs disclosed in U.S. Pat. No. 5,011,691, the disclosure of which is incorporated herein by reference (e.g., COP-1, COP-3, COP-4, COP-5, COP-7, and COP-16).

In other preferred embodiments, the OP/BMP morphogens useful herein include proteins which comprise an amino acid sequence sharing at least 70% amino acid sequence “homology” and, preferably, 75% or 80% homology with the C-terminal seven cysteine domain present in the active forms of human OP-1 (i.e., residues 330-431, as shown in SEQ ID NO: 2 of U.S. Pat. No. 5,266,683) or GDF-5. In other preferred embodiments, the OP/BMP morphogens useful herein include proteins which comprise an amino acid sequence sharing at least 60% amino acid sequence identity and, preferably, 65% or 70% identity with the C-terminal seven cysteine domain present in the active forms of human OP-1 or GDF-5. Thus, a candidate amino acid sequence can be aligned with the amino acid sequence of the C-terminal seven cysteine domain of human OP-1 using the method of Needleman el al., J. Mol. Biol. 48:443-453 (1970), implemented conveniently by computer programs such as the Align program (DNAstar, Inc.). As will be understood by those skilled in the art, homologous or functionally equivalent sequences include functionally equivalent arrangements of the cysteine residues within the conserved cysteine skeleton, including amino acid insertions or deletions which alter the linear arrangement of these cysteines, but do not materially impair their relationship in the folded structure of the dimeric protein, including their ability to form such intra- or inter-chain disulfide bonds as may be necessary for biological activity. Therefore, internal gaps and amino acid insertions in the candidate sequence are ignored for purposes of calculating the level of amino acid sequence homology or identity between the candidate and reference sequences.

“Amino acid sequence homology” is understood herein to include both amino acid sequence identity and similarity. Thus, as used herein, a percentage “homology” between two amino acid sequences indicates the percentage of amino acid residues which are identical or similar between the sequences. “Similar” residues are “conservative substitutions” which fulfill the criteria defined for an “accepted point mutation” in Dayhoffel al., Atlas of Protein Sequence and Structure Vol. 5 (Suppl. 3), pp. 354-352 (1978), Natl. Biomed. Res. Found., Washington, D.C. Thus, “conservative amino acid substitutions” are residues that are physically or functionally similar to the corresponding reference residues, having similar size, shape, electric charge, and/or chemical properties such as the ability to form covalent or hydrogen bonds, or the like. Examples of conservative substitutions include the substitution of one amino acid for another with similar characteristics, e.g., substitutions within the following groups. (a) valine, glycine, (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. The term “conservative substitution” or “conservative variation” also includes the use of a substituted amino acid in place of an unsubstituted parent amino acid in a given polypeptide chain, provided that the resulting substituted polypeptide chain has biological activity useful in the present invention.

The OP/BMP morphogens of the invention are characterized by biological activities which may be readily ascertained by those of ordinary skill in the art.

The OP/BMP morphogens contemplated herein can be expressed from intact or truncated genomic or cDNA or from synthetic DNAs in prokaryotic or eukaryotic host cells. The dimeric proteins can be isolated from the culture media and/or refolded and dimerized in vitro to form biologically active preparations. Heterodimers can be formed in vitro by combining separate, distinct polypeptide chains. Alternatively, heterodimers can be formed in a single cell by co-expressing nucleic acids encoding separate, distinct polypeptide chains. See, for example, WO93/09229, or U.S. Pat. No. 5,411,941, for several exemplary recombinant heterodimer protein production protocols. Currently preferred host cells include, without limitation, prokaryotes including E. coli, or eukaryotes including yeast such as Saccharomyces, insect cells, or mammalian cells, such as CHO, COS or BSC cells. One of ordinary skill in the art will appreciate that other host cells can be used to advantage. Detailed descriptions of the proteins useful in the practice of this invention, including how to make, use and test them for activity, are disclosed in numerous publications, including U.S. Pat. Nos. 5,266,683 and 5,011,691, the disclosures of which are herein incorporated by reference.

In some embodiments, the growth factor is GDF-5. When GDF-5 is selected as the growth factor, it may be combined with a PLGA carrier.

In some embodiments, the therapeutic agent is an anti-tumor therapeutic, such as BCNU. When BCNU is selected as the anti-tumor therapeutic, is may be combined with a PLGA carrier. Techniques for providing BCNU in a PLGA carrier are disclosed in Chae, Int. J. Pharm., July 2005, 14 (e-pub).

The present inventors have further developed inventions for treating neurodegenerative disease with a composite comprising a microbubble containing a high affinity for soluble beta amyloid protein.

In preferred embodiments, the composite in administered intrathecally (such as through a lumbar puncture) and then buoyantly lifted through the spinal CSF and into the cranium to the site of the brain injury. Once sited at the location of the injury, the compound having a high affinity for soluble beta amyloid protein is released from the composite and ameliorates the disease.

In some embodiments, the compound having a high affinity for soluble beta amyloid protein is VEGF. Yang, J. Neurochem., April 2005, 93(1) 118-127 reports that VEGF provides neuroprotection in AD by binding to beta amyloid protein.

In addition to VEGF, there are other molecules that have high affinity binding to beta amyloid protein. These include gelsolin and GM I (Matsuoka, J. Neurosci., January 2003, 1., 23(1) 29-33), and Congo Red, Chrysamine and Thiflavin S (Lee, Neurobiol. Aging, Novenber.December 2002, 23(6) 1039-1042).

The present inventors have further developed inventions for treating neurodegenerative disease with a composite comprising a microbubble containing an anti-inflammatory compound.

In preferred embodiments, the composite in administered intrathecally (such as through a lumbar puncture) and then buoyantly lifted through the spinal CSF and into the cranium to the site of the brain injury. Once sited at the location of the injury, the anti-inflammatory compound is released from the composite and ameliorates the disease.

In some embodiments, the anti-inflammatory compound is an antagonist is capable of specifically inhibiting a pro-inflammatory cytokine, termed a “high specificity cytokine antagonist, or “HSCA”). In some embodiments, the antagonist is capable of specifically inhibiting a pro-inflammatory cytokine selected from the group consisting of TNF-α, an interleukin (preferably, IL-1, II-6 and IL-8), FAS, an FAS ligand, and IFN-gamma. In some embodiments, the HSCA inhibits the cytokine by preventing its production. In some embodiments, the HSCA inhibits the cytokine by binding to a membrane-bound cytokine. In others, the HSCA inhibits the cytokine by binding to a solubilized cytokine. In some embodiments, the HSCA inhibitor inhibits the cytokine by both binding to membrane bound cytokines and to solubilized cytokine. In some embodiments, the HSCA is a monoclonal antibody (“InAb”). The use of mAbs is highly desirable since they bind specifically to a certain target protein and to no other proteins. In some embodiments, the HSCA inhibits the cytokine by binding to a natural receptor of the target cytokine.

In some embodiments, the HSCA inhibits the cytokine by preventing its production. One example thereof is an inhibitor of p38 MAP kinase. In some embodiments, the TNF inhibitor inhibits the TNF by binding to membrane bound TNF in order to prevent its release from membrane. In others, the TNF inhibitor inhibits the TNF by binding to solubilized TNF. One example thereof is etanercept. In some embodiments, the TNF inhibitor inhibits the TNF by both binding to membrane bound TNF and to solubilized TNF. One example thereof is infliximab. In some embodiments, the HSCA inhibits the cytokine by binding to a natural receptor of the target cytokine.

Any method of the present invention can comprise administering an effective amount of a composition or pharmaceutical composition comprising at least one anti-TNF-α antibody to a cell, tissue, organ, animal or patient in need of such modulation, treatment or therapy. Such a method can optionally further comprise co-administration or combination therapy for treating such diseases or disorders, wherein the administering of said at least one anti-TNF-α antibody, specified portion or variant thereof, further comprises administering, before concurrently, and/or after, at least one selected from at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-I or TBP-II), nerelimonmab, infliximab, enteracept (Enbrel™), adalimulab (Humira™), CDP-571, CDP-870, afelimomab, lenercept, and the like), an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, domase alpha (Pulmozyme), a cytokine or a cytokine antagonist. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); Nursing 2001 Handbook of Drugs, 21^{st} edition, Springhouse Corp., Springhouse, Pa., 2001; Health Professional's Drug Guide 2001, ed., Shannon, Wilson, Stang, Prentice-Hall, Inc, Upper Saddle River, N.J. each of which references are entirely incorporated herein by reference.

TNF antagonists suitable for compositions, combination therapy, co-administration, devices and/or methods of the present invention (further comprising at least one anti body, specified portion and variant thereof, of the present invention), include, but are not limited to, anti-TNF antibodies (e.g., at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-I or TBP-II), nerelimonmab, infliximab, enteracept (Enbrel™), adalimulab (Humira™), CDP-571, CDP-870, afelimomab, lenercept, and the like), antigen-binding fragments thereof, and receptor molecules which bind specifically to TNF; compounds which prevent and/or inhibit TNF synthesis, TNF release or its action on target cells, such as thalidomide, tenidap, phosphodiesterase inhibitors (e.g, pentoxifylline and rolipram), A2b adenosine receptor agonists and A2b adenosine receptor enhancers; compounds which prevent and/or inhibit TNF receptor signalling, such as mitogen activated protein (MAP) kinase inhibitors; compounds which block and/or inhibit membrane TNF cleavage, such as metalloproteinase inhibitors; compounds which block and/or inhibit TNF activity, such as angiotensin converting enzyme (ACE) inhibitors (e.g., captopril); and compounds which block and/or inhibit TNF production and/or synthesis, such as MAP kinase inhibitors.

As used herein, a “tumor necrosis factor antibody,” “TNF antibody,” “TNFα antibody,” or fragment and the like decreases, blocks, inhibits, abrogates or interferes with TNFα activity in vitro, in situ and/or preferably in vivo. For example, a suitable TNF human antibody of the present invention can bind TNFα and includes anti-TNF antibodies, antigen-binding fragments thereof, and specified mutants or domains thereof that bind specifically to TNFα. A suitable TNF antibody or fragment can also decrease block, abrogate, interfere, prevent and/or inhibit TNF RNA, DNA or protein synthesis, TNF release, TNF receptor signaling, membrane TNF cleavage, TNF activity, TNF production and/or synthesis.

Chimeric antibody cA2 consists of the antigen binding variable region of the high-affinity neutralizing mouse anti-human TNFα IgG1 antibody, designated A2, and the constant regions of a human IgG1, kappa immunoglobulin. The human IgG1 Fc region improves allogeneic antibody effector function, increases the circulating serum half-life and decreases the immunogenicity of the antibody. The avidity and epitope specificity of the chimeric antibody cA2 is derived from the variable region of the murine antibody A2. In a particular embodiment, a preferred source for nucleic acids encoding the variable region of the murine antibody A2 is the A2 hybridoma cell line.

Chimeric A2 (cA2) neutralizes the cytotoxic effect of both natural and recombinant human TNFα in a dose dependent manner. From binding assays of chimeric antibody cA2 and recombinant human TNFα, the affinity constant of chimeric antibody cA2 was calculated to be 1.04×10¹⁰ M⁻¹. Preferred methods for determining monoclonal antibody specificity and affinity by competitive inhibition can be found in Harlow, et al., antibodies: A Laboratory Manual, Cold Spring Harbor Laboratory Press, Cold Spring Harbor, New York, 1988; Colligan et al., eds., Current Protocols in Immunology, Greene Publishing Assoc. and Wiley Interscience, New York, (1992-2000); Kozbor et al., Immunol. Today, 4:72-79 (1983); Ausubel et al., eds. Current Protocols in Molecular Biology, Wiley Interscience, New York (1987-2000); and Muller, Meth. Enzymol., 92:589-601 (1983), which references are entirely incorporated herein by reference. In a particular embodiment, murine monoclonal antibody A2 is produced by a cell line designated c134A. Chimeric antibody cA2 is produced by a cell line designated c168A.

Additional examples of monoclonal anti-TNF antibodies that can be used in the present invention are described in the art (see, e.g., U.S. Pat. No. 5,231,024; Möller, A. et al., Cytokine 2(3):162-169 (1990); U.S. application Ser. No. 07/943,852 (filed Sep. 11, 1992); Rathjen et al., International Publication No. WO 91/02078 (published Feb. 21, 1991); Rubin et al., EPO Patent Publication No. 0 218 868 (published Apr. 22, 1987); Yone et al., EPO Patent Publication No. 0 288 088 (Oct. 26, 1988); Liang, et al., Biochem. Biophys. Res. Comm. 137:847-854 (1986); Meager, et al., Hybridoma 6:305-311 (1987); Fendly et al., Hybridoma 6:359-369 (1987); Bringman, et al., Hybridoma 6:489-507 (1987); and Hirai, et al., J. Immunol. Meth. 96:57-62 (1987), which references are entirely incorporated herein by reference).

TNF Receptor Molecules. Preferred TNF receptor molecules useful in the present invention are those that bind TNFα with high affinity (see, e.g., Feldmann et al., International Publication No. WO 92/07076 (published Apr. 30, 1992); Schall et al., Cell 61:361-370 (1990); and Loetscher et al., Cell 61:351-359 (1990), which references are entirely incorporated herein by reference) and optionally possess low immunogenicity. In particular, the 55 kDa (p55 TNF-R) and the 75 kDa (p75 TNF-R) TNF cell surface receptors are useful in the present invention. Truncated forms of these receptors, comprising the extracellular domains (ECD) of the receptors or functional portions thereof (see, e.g., Corcoran et al., Eur. J. Biochem. 223:831-840 (1994)), are also useful in the present invention. Truncated forms of the TNF receptors, comprising the ECD, have been detected in urine and serum as 30 kDa and 40 kDa TNFα inhibitory binding proteins (Engelmann, H. et al., J. Biol. Chem. 265:1531-1536 (1990)). TNF receptor multimeric molecules and TNF immunoreceptor fusion molecules, and derivatives and fragments or portions thereof, are additional examples of TNF receptor molecules which are useful in the methods and compositions of the present invention. The TNF receptor molecules which can be used in the invention are characterized by their ability to treat patients for extended periods with good to excellent alleviation of symptoms and low toxicity. Low immunogenicity and/or high affinity, as well as other undefined properties, can contribute to the therapeutic results achieved.

TNF receptor multimeric molecules useful in the present invention comprise all or a functional portion of the ECD of two or more TNF receptors linked via one or more polypeptide linkers or other nonpeptide linkers, such as polyethylene glycol (PEG). The multimeric molecules can further comprise a signal peptide of a secreted protein to direct expression of the multimeric molecule. These multimeric molecules and methods for their production have been described in U.S. application Ser. No. 08/437,533 (filed May 9, 1995), the content of which is entirely incorporated herein by reference.

TNF immunoreceptor fusion molecules useful in the methods and compositions of the present invention comprise at least one portion of one or more immunoglobulin molecules and all or a functional portion of one or more TNF receptors. These immunoreceptor fusion molecules can be assembled as monomers, or hetero- or homo-multimers. The immunoreceptor fusion molecules can also be monovalent or multivalent. An example of such a TNF immunoreceptor fusion molecule is TNF receptor/IgG fusion protein. TNF immunoreceptor fusion molecules and methods for their production have been described in the art (Lesslauer et al., Eur. J. Immunol. 21:2883-2886 (1991); Ashkenazi et al., Proc. Natl. Acad. Sci. USA 88:10535-10539 (1991); Peppel et al., J. Exp. Med. 174:1483-1489 (1991); Kolls et al., Proc. Natl. Acad. Sci. USA 91:215-219 (1994); Butler et al., Cytokine 6(6):616-623 (1994); Baker et al., Eur. J. Immunol. 24:2040-2048 (1994); Beutler et al., U.S. Pat. No. 5,447,851; and U.S. application Ser. No. 08/442,133 (filed May 16, 1995), each of which references are entirely incorporated herein by reference). Methods for producing immunoreceptor fusion molecules can also be found in Capon et al., U.S. Pat. No. 5,116,964; Capon et al., U.S. Pat. No. 5,225,538; and Capon et al., Nature 337:525-531 (1989), which references are entirely incorporated herein by reference.

A functional equivalent, derivative, fragment or region of TNF receptor molecule refers to the portion of the TNF receptor molecule, or the portion of the TNF receptor molecule sequence which encodes TNF receptor molecule, that is of sufficient size and sequences to functionally resemble TNF receptor molecules that can be used in the present invention (e.g., bind TNFα with high affinity and possess low immunogenicity). A functional equivalent of TNF receptor molecule also includes modified TNF receptor molecules that functionally resemble TNF receptor molecules that can be used in the present invention (e.g., bind TNFα with high affinity and possess low immunogenicity). For example, a functional equivalent of TNF receptor molecule can contain a “SILENT” codon or one or more amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., Current Protocols in Molecular Biology, Greene Publishing Assoc. and Wiley-Interscience, New York (1987-2000).

In some embodiments, the anti-inflammatory compound comprises α-MSH. PLGA may be a carrier for α-MSH. Bhardwaj, Pharm. Res., May 2000 17(5) 593-9 teaches the release of α-MSH from PLGA over 24 hours.

Among the therapeutic agents which may be micro encapsulated and administered into the cerebrospinal fluid according to the present invention can be, preferably, anti-inflammatory agents. As used herein the term “anti-inflammatory agents” refers to any agent which possesses the ability to reduce or eliminate cerebral edema (fluid accumulation), cerebral ischemia, or cell death caused by traumatic brain injury (TBI) or stroke. Categories of anti-inflammatory agents include:

a) Free radical scavengers and antioxidants, which act to chemically alter (dismutate) or scavenge the different species of oxygen radicals produced due to ischemic and trauma associated events. Unless dismutated or scavenged, these highly reactive free radicals cause the peroxidation (breakdown) of cell membrane phospholoipids (lipid peroxidation) and the oxidation of cellular proteins and nucleic acids leading to severe tissue damage and death of neurons. Examples of such drugs are superoxide dismutase, catalase, nitric oxide, mannitol, allopurinol, dimethyl sulfoxide.

b) Nonsteroidal anti-inflammatory drugs (NSAIDS), which act to reduce cell migration, caused by ischemic and trauma associated events, and therefore slow down edema formation, as well as provide pain relief. Examples of such drugs are aspirin, acetaminophen, indomethacin, ibuprofen.

c) Steroidal anti-inflammatory agents (Glucocorticoids, Hormones), which can enhance or prevent the immune and inflammatory process and inhibit lipid peroxidation as seen in the events that occur during oxygen radical formation. Examples of such drugs are cortisone, prednisone, prednisolone, dexamethasone. The most well known of these is dextramethasone which has been used for reduction of cerebral edema after TBI.

d) Calcium channel blockers, which act to prevent excess calcium from entering the cell during cerebral ischemia. Some of these drugs also have other beneficial effects on increasing cerebral blood flow to the brain. Examples of such drugs are nimodipine, nifedipine, verapamil, nicardipine.

e) NMDA antagonists, which block the NMDA receptor site for glutamate, a neurotransmitter released excessively during ischemia. Excess glutamate can activate the NMDA receptors leading to increase firing which will in turn cause cell swelling and an influx of calcium leading to cell death. Examples of such drugs are magnesium sulfate and dextromethorphan, actually an opioid analogue.

f) citicholine. Citicholine prevents toxic free fatty acid accumulation, promotes recovery of brain function by providing two components, cytidine and choline, required in the formation of nerve cell membrane, promoting the synthesis of acetylcholine, a neurotransmitter associated with cognitive function.

g) Stress proteins, such as hsp70, hsp 27, and heme oxygenase.

h) recombinant glutamate receptors, such as GluR1, and

i) BMPs, such as rhGDF-5.

The therapeutic agents can be used alone or in combination with one or more other therapeutic agents to achieve a desired effect.

Preferred bioresorbable materials which can be used to make the microbubbles of the present invention include bioresorbable polymers or copolymers, preferably selected from the group consisting of hydroxy acids, (particularly lactic acids and glycolic acids; caprolactone; hydroxybutyrate; dioxanone; orthoesters; orthocarbonates; and aminocarbonates). Preferred bioresorbable materials also include natural materials such as chitosan, collagen, cellulose, fibrin, hyaluronic acid; fibronectin, and mixtures thereof. However, synthetic bioresorbable materials are preferred because they can be manufactured under process specifications which insure repeatable properties. (see http://www.emedicine.com/derm/topic825.htm for listing of nonresorbable materials)

A variety of bioabsorbable polymers can be used to make the microbubbles of the present invention. Examples of suitable biocompatible, bioabsorbable polymers include but are not limited to polymers selected from the group consisting of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biomolecules (i.e., biopolymers such as collagen, elastin, bioabsorbable starches, etc.) and blends thereof. For the purpose of this invention aliphatic polyesters include, but are not limited to, homopolymers and copolymers of lactide (which includes lactic acid, D-,L- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, δ-valerolactone, β-butyrolactone, χ-butyrolactone, ε-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one, 2,5-diketomorpholine, pivalolactone, χ,χ-diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one and polymer blends thereof. Poly(iminocarbonates), for the purpose of this invention, are understood to include those polymers as described by Kemnitzer and Kohn, in the Handbook of Biodegradable Polymers, edited by Domb, et. al., Hardwood Academic Press, pp. 251-272 (1997). Copoly(ether-esters), for the purpose of this invention, are understood to include those copolyester-ethers as described in the Journal of Biomaterials Research, Vol. 22, pages 993-1009, 1988 by Cohn and Younes, and in Polymer Preprints (ACS Division of Polymer Chemistry), Vol. 30(1), page 498, 1989 by Cohn (e.g. PEO/PLA). Polyalkylene oxalates, for the purpose of this invention, include those described in U.S. Pat. Nos. 4,208,511; 4,141,087; 4,130,639; 4,140,678; 4,105,034; and 4,205,399. Polyphosphazenes, co-, ter- and higher order mixed monomer-based polymers made from L-lactide, D,L-lactide, lactic acid, glycolide, glycolic acid, para-dioxanone, trimethylene carbonate and ε-caprolactone such as are described by Allcock in The Encyclopedia of Polymer Science, Vol. 13, pages 31-41, Wiley Intersciences, John Wiley & Sons, 1988 and by Vandorpe, et al in the Handbook of Biodegradable Polymers, edited by Domb, et al, Hardwood Academic Press, pp. 161-182 (1997). Polyanhydrides include those derived from diacids of the form HOOC—C₆H₄—O—(CH₂)ₘ-O—C₆H₄—COOH, where m is an integer in the range of from 2 to 8, and copolymers thereof with aliphatic alpha-omega diacids of up to 12 carbons. Polyoxaesters, polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213; 5,700,583; and 5,859,150. Polyorthoesters such as those described by Heller in Handbook of Biodegradable Polymers, edited by Domb, et al, Hardwood Academic Press, pp. 99-118 (1997).

Preferably, the bioresorbable material is selected from the group consisting of poly(lactic acid) (“PLA”) and poly(glycolic acid)(“PGA”), and PLGA copolymers thereof.

In some embodiments, albumin can be used to make the microbubbles of the present invention

In some embodiments, the microbubbles containing the therapeutic agent are administered intrathecally.

In some embodiments, the microbubbles are injected into the CSF through a sterile lumbar puncture and are allowed to float up through the spinal column and into the cerebroventricular system. Once the microbubbles are in the patient's brain, the microbubbles may be subjected to ultrasound to explode the microbubbles and thereby release the therapeutic agent into the CSF.

In some embodiments, the microbubbles are injected into the CSF at or near the cisterna magnum.

In other embodiments, the microbubble containing the therapeutic agent is injected into a cerebral vein and the patient's head is oriented so that the microbubble travels upwards and in a retrograde manner to reach the CSF. Inoue, Ca. J. Neurol. Sci., August 1996, 23(3)175-83 investigated the retrograde infusion of the cerebral vein with an anti-oxidant (LY231617) in a rat middle cerebral artery occlusion model, and reported that there was a significant increase in local cerebral blood flow, a significant improvement in BBB permeability, and significantly reduced ischemic damage at seven hours post-MCA occlusion.

In some embodiments, the retrograde infusion is accomplished by first creating a small hole in the patient's skull and inserting a catheter into a cerebral vein in the patient's vasculature.

In some retrograde infusion embodiments, the microbubbles containing the therapeutic agent are injected into an emissary vein in the patient's scalp and the patient's head is oriented so that the microbubbles float upwards and in a retrograde manner through connecting veins to reach the CSF.

In some embodiments, the emissary vein is the parietal emissary vein. This vein, which enters the skull through the parietal foramen, connects with the superior saggital sinus.

In some embodiments, the emissary vein is the mastoid emissary vein. This vein, which enters the skull through the mastoid foramen, connects the occipital vv with the sigmoid sinus.

In some embodiments, the emissary vein is the occipital emissary vein.

In some embodiments, the emissary vein is the condyloid emissary vein. This vein, which enters the skull through the condylar canal, connects the suboccipital plexus vv with the sigmoid sinus.

In some embodiments, the microbubbles are injected into the lymphatic system of the patient and the patient is oriented so that the microbubble float upwards through the cribriform plate and into the brain. The literature reports that a significant percentage of CSF exits the patient's brain through the cribriform plate, just above the nasal cavity, and is absorbed by cervical lymph nodes. Once in the ventricles are the patient's brain, the microbubbles may be subjected to ultrasound to explode the microbubbles and thereby release the therapeutic agent into the CSF.

Therefore, in some embodiments, the microbubbles of the present invention are injected into the lymphatic system that drains the cribriform plate. In some embodiments thereof, the microbubbles of the present invention are injected into the cervical lymph nodes, preferably in a lymphatic vessel just above a cervical lymph node.

In embodiments of the present invention in which the therapeutic agent resides within the porosity of the honeycombed microbubble, it may be advantageous to force the release of the therpauetic agent by destroying the microbubbles in a predetermined manner once they have floated up to the cranium. In preferred embodiments thereof, this may be achieved by applying an effective amount of ultrasound to the microbubbles.

As a consequence of improvements in ultrasound technology, the destruction of cerebrovascular microbubbles by transcranial ultrasound in human patients has been demonstrated in the art. See, for example, Culp, Stroke, October 2004, 35,10,2407-10; Kern, Stroke, July 2004, 35(7) 1665-70; Eyding, J. Neuroimaging, April 2004, 14, 2, 143-9; Seidel, Ultrasound Med. Biol., February 2002, 28,2,183-9. Therefore, it is reasonable to believe that the destruction of microbubbles present in the CSF by transcranial unfocused ultrasound in human patients is easily within the grasp of the skilled artisan.

In some embodiments, the ultrasound is provided by transcranial application of focused ultrasound. This allows the clinician to destroy the microbubbles in a select region of the cranial CSF.

Historically, transcranial focused ultrasound therapies have been avoided due to the high distortion and energy absorption associated with the bone of the skull. Recently however, there have been numerous reports in the literature that these problems have been solved. In particular, new transcerebral ultrasound technology is able to precisely and accurately heat selective portions of the brain. In one particular report, Hynynen, Magnetic Resonance in Medicine, 52:100-107(2004), the investigators were able to produce a 39° C. degree peak in an experimental set up comprising an exposed rat brain located within a water-filled human skull. According to the authors, recent advances in transducer, amplifier and medical imaging technology as well as progress in ultrasound modeling have increased the feasibility of using focused ultrasound for noninvasive brain therapy. Therefore, it is reasonable to expect that straightforward modification of this apparatus can provide for its use in patients.

In some embodiments, the transcranial ultrasound technology disclosed in U.S. Pat. No. 6,770,031, entitled “Ultrasound Therapy” (Hynynen), the specification of which is incorporated by reference in its entirety, is used. Other articles disclosing suitable transcranial ultrasound technology for use in the present invention include Sun, J. Acoust. Soc. Am., April 1999, 105(4) 2519-27; Sun, J. Acoust. Soc. Am., September 1998, 104(3 Pt. 1):1705-15; Connor, IEEE Trans. Biomed. Eng. October 2004 51(10) 1693-706; Clement, Phys. Med. Biol. Apr. 21, 2002; 47(8), 1219-36; Hynenen, Ultrasound in Med. & Biol. 24(2) 275-283 (1998); Nynynen, Neuroimage., January 2005, 1, 24(1) 12-20; Clement, Phys. Med. Biol., December 2000 45(12) 3707-19; Clement, Phys. Med. Biol. April 2000 45(4) 1071-83; the specifications of which are incorporated by reference in their entirety.

In some embodiments, the ultrasound is applied epidurally. For the purposes of the present invention, in an “epidural” application of ultrasound, the ultrasound transducer is placed just outside the dura and thereby avoids transmission through the skull. This is advantageous because ultrasound couples to the skull, thereby causing heating of the skull, attenuation of the ultrasound intensity, and distortion of the ultrasound waves. Epidural application of ultrasound avoids heating the skull and allows for the focused presentation of ultrasound to the cancerous region of the brain tissue.

In some transdural embodiments, a portion of the skull is removed to form a bore in the skull. The epidural ultrasound is then transmitted through the bore to the brain tissue.

In some embodiments, a prosthetic, ultrasound transmissible window is placed in the bore in the skull. The literature has reported that such windows allow the surgeon to perform scanned, focused ultrasound treatments of brain tumors. Tobias, Med. Phys. March-April 1987, 14,2,228-34. The placement of the window in the bore allows the surgeon to essentially non-invasively treat the cancer on a repeated basis after the initial surgery to implant the window.

The window material is preferably selected from the group consisting of polyethylene, polystyrene, acrylic, and PMMA. More preferably, the window material is polyethylene.

In some embodiments, there is provided an ultrasonic cutter for cutting a small bore in the skull. The vibrations produced by an ultrasonic cutter can cut through bone, but avoid cutting through more flexible tissue such as the dura. Accordingly, the ultrasonic cutter may be used to bore a path for epidural placement of an ultrasound transducer.

In some embodiments, the ultrasound is passed through the cribriform plate in the prefrontal cortex. Since the cribriform plate (which separates the brain from the nasal cavity) is very thin and highly porous (˜50 areal %), it is believed that ultrasound will pass therethrough with very little attenuation or distortion. Accordingly, it is believed that focused ultrasound can be intranasally applied to microbubbles present adjacent an infarct in the prefrontal cortex without having to create a bore in the intervening bone, resulting in the destruction of the microbubbles.

In some embodiments, the application of ultrasound energy to the microbubbles is carried out at a frequency of about 1 MHz and a power of about 2.2 W/cm². In some embodiments, the ultrasound transducer operates at a resonance frequency of about 1.13 MHz. See Hwang, supra.

According to Dijkmans, Eur. J. Echocariography, 2004, 5, 245-256, high pressure ultrasound having a mechanical index (MI) greater than 1.0 causes forced expansion and compression of the microbubbles, leading to bubble destruction. Therefore, in preferred embodiments, the applied ultrasound produces a mechanical index of greater than 1.0, more preferably at least 1.5, more preferably at least 2.0.

Preferably, the acoustic pressure resulting from the explosion of the microbubbles is between about 1 MPa and about 6.5 MPa, more preferably between 3.35 MPa and 6.5 MPa.

The composites of the present invention may be used to treat neurodegenerative diseases such as traumatic brain injury (TBI), Alzheimer's Disease, Parkinson's Disease, stroke and Multiple Sclerosis.

In some embodiments, the composites of the present invention may be used to treat Subarachnoid Hemorrhage (SAH). Subarachnoid hemorrhage occurs when blood collects beneath the arachnoid mater membrane that lies between the brain and the skull. SAH is a great cause for concern because it often leads to cerebrovasospasm and death.

The primary cause of SAH appears to be traumatic brain injury. It has been estimated that between between 23% and 39% of the 373,000 people who are hospitalized with TBI each year in the United States have SAH. In addition, it is estimated that there are about 23,000 cases/year of SAH in the United States associated with stroke.

In recent years, there have been a number of therapies for SAH that have involved the intracisternal injection of therapeutic agents in order to dissolve the blood clot and thereby lower the chances for cerebrovasospasm. However, intracisternal injections are often very complicated procedures.

The present inventors have appreciated that intrathecal injection of buoyant microbubbles can deposit the therapeutic agent in the cisterna magna (and thereby mimic the results of an intracisternal injection). In addition, intrathecal injection of buoyant microbubbles can likely deposit the therapeutic agent beneath the circle of Willis. Therefore, intrathecal injection of buoyant microbubbles can deposit the therapeutic agent at approximately the site of the SAH, where they can be exploded to release the therapeutic agent.

In some embodiments, intrathecal microbubbles containing a thrombolytic agent is used to treat the SAH. In some embodiments, the thrombolytic agent is used to treat the SAH is selected from the group consisting of tPA, urokinase, and a thrombin inhibitor.

In some embodiments, intrathecal microbubbles containing tissue plasminogen activator (tPA) are used to treat the SAH. Molina, Stroke, 2006, 37, 425-429, reports that an infusion of tPA and microbubbles accelerate clot lysis.

In some embodiments, intrathecal microbubbles containing urokinase are used to treat the SAH. Hamada, Stroke, 2000, 31:2141-8, reports that intracisternal injection of urokinase in patients with recently ruptured aneurysms is a safe and reasonable means of preventing vasospasms and may result in improved outcomes. Hamada, Stroke, 2003, 34:2549-2554, reports that intracisternal injection of urokinase in patients with recently ruptured aneurysms significantly reduced the occurance of symptomatic vasospasm and resulted in a lower rate of permanent neurological deficits.

In some embodiments, intrathecal microbubbles containing a thrombin inhibitor are used to treat the SAH. In some embodiments, the thrombin inhibitor is hirudin. Kudo, Cerebrovascular Diseases, 2000, 10:424-430, reports that implantation of collagen devices containing hirudin lowered the reduction in the diameter of the basilar artery in canines who had autologous blood implanted in the cisterna magna. Kudo concludes that the thrombin inhibitor hirudin ameliorated the physiological and histological effects of vasospasm following SAH in a single hemorrhage canine model.

In some embodiments, intrathecal microbubbles containing a vasodilator are used to treat the SAH. In some embodiments, the vasodilator is nicardipine. Kasuya, Neurosurgery, 56, 895-902, 2005, reports that placing prolonged release nicardipine implanted in the cisterna of the internal carotid artery of patients with SAH may decrease the incidence of delayed ischemic neurological deficit. Kasuya, Stroke, 2002, 33, 1011-1015, reports that placing prolonged release nicardipine implanted in the cisterna of the internal carotid artery of patients with SAH completely prevented vasospasm for the arteries with thick cistern clots.

In some embodiments, intrathecal microbubbles containing a nitric oxide donor are used to treat the SAH. In some embodiments, the nitric oxide donor is diethylene-triamine/NO. Gabikian, Stroke, 2002, 33, 2681-6 reports that implanting sustained release diethylene-triamine/NO implants in the subarachnoid space after injection of autologous blood in the cisterna magna of rabbits prevented vasospasm in the rabbit basilar artery. Pradilla, Neurosurgery, 55, 1393-1400,2004, reports that implanting sustained release diethylene-triamine/NO implants in the cisterna magna after injection of autologous blood in the cisterna magna of rabbits prevented vasospasm in rabbits.

In some embodiments, intrathecal microbubbles containing a calcium antagonist are used to treat the SAH.

In some embodiments, the composites of the present invention may be used to treat stroke, and in particular to enhance the functional neurological recovery after stroke. It is further believed that the intrathecal delivery of buoyant microbubbles will find special application in promoting functional recovery after stroke.

In particular, it has been reported in the literature that intracisternal injection of BMP-7 (OP-1) into the cisterna magna promotes functional recovery of neurons after stroke in rats. Ren, Neuropharmacology, 39, 2000, 860-865 and Liu, Brain Research 905, 2001, 81-90.

Because buoyant intrathecal microbubbles likely rise up into the cisterna magna and just below the Circle of Willis, it is likely that they would be specially situated to treat neurons that have been damaged by stroke. Therefore, there is provided a simple lumbar puncture to inject a BMP (especially, GDF-5)—containing buoyant microbubbles into these areas, wherein the microbubbles are then exploded with ultrasound. This method of treatment would be much less invasive for the patient as compared to an intracisternal injection.

In some stroke embodiments, GDNF is added to the BMP.

We claim:

1. A method of delivering a therapeutic drug to a brain of a patient, comprising the step of:
a) intrathecally administering a plurality of microbubbles comprising a therapeutic agent and a carrier to the patient,
b) allowing the composite to rise into the cranium, and
c) applying ultrasound to the cranium to explode the microbubbles.

2. The method of claim 1 wherein the microbubbles have a size of not more than 100 μm.

3. The method of claim 1 wherein the microbubbles have a size of not more than 50 μm.

4. The method of claim 1 wherein the microbubbles have a size of not more than 20 μm.

5. The method of claim 1 wherein the microbubbles have a size of not more than 5 μm.

6. The method of claim 1 wherein the microbubbles have a density of less than 0.6 g/cc.

7. The method of claim 1 wherein the microbubbles have a density of less than 0.4 g/cc.

8. The method of claim 1 wherein the microbubbles comprise a polymeric structure comprising:
i) an outer wall section comprising a carrier matrix, and
ii) a central porous honeycomb section.

9. The method of claim 8 wherein the outer wall is substantially non-porous.

10. The method of claim 8 wherein the microbubbles further comprise:
iii) a therapeutic agent, wherein the therapeutic agent is encapsulated within the central porous section.

11. The method of claim 8 wherein the microbubbles further comprise:
iii) a therapeutic agent, wherein the therapeutic agent is embedded in the polymeric structure.

12. The method of claim 1 wherein the therapeutic agent comprises EPO.

13. The method of claim 1 wherein the therapeutic agent comprises magnesium.

14. The method of claim 1 wherein the therapeutic agent comprises a thrombolytic agent.

15. A method of delivering a therapeutic drug to a brain of a patient, comprising the step of:
a) intrathecally administering to the patient a composite having a density of less than 1 g/cc, wherein the composite comprises a carrier and a therapeutic agent,
b) allowing the composite to rise into the cranium, and
c) applying ultrasound to the cranium to explode the microbubbles.

16. A composite comprising:
a) an effective amount of magnesium that treats a neurodegenerative disease of the brain, and
b) a carrier encapsulating the effective amount of magnesium,
wherein the composite has a density of less than 1 g/cc and a size of not more than 10 μm.

17. The composite of claim 16 wherein the carrier is in the form of a microbubble.

18. The composite of claim 16 wherein the magnesium comprises magnesium sulfate.

19. The composite of claim 16 wherein the magnesium comprises magnesium chloride.

20. The composite of claim 16 having a density of less than 0.6 g/cc.
